(19) United States
(12) Patent Application Publication
(10) Pub. No.: US 2020/0273566 A1
(43) Pub. Date: Aug. 27, 2020

(54) SHARING OF HEALTH-RELATED DATA BASED ON DATA EXPORTED BY EAR-WEARABLE DEVICE

(71) Applicant: Starkey Laboratories, Inc., Eden Prairie, MN (US)

(72) Inventors: Achintya Kumar Bhowmik, Cupertino, CA (US); David A. Fabry, Eden Prairie, MN (US); Kyle Walsh, Minneapolis, MN (US); Thomas Scheller, Eden Prairie, MN (US); Christopher L. Howes, Eden Prairie, MN (US)

(21) Appl. No.: 16/797,894

(22) Filed: Feb. 21, 2020

Related U.S. Application Data
(60) Provisional application No. 62/809,264, filed on Feb. 22, 2019.

(51) Int. Cl. G16H 40/63; H04R 25/00

(52) U.S. Cl. CPC G16H 40/63; H04R 25/554; H04R 2225/55; H04R 2225/023; H04R 2225/021; H04R 2225/025

(57) ABSTRACT

A computing system receives sharing preference data. The sharing preference data indicate, for each respective health-related data type of a plurality of health-related data types: (i) whether a user of an ear-wearable device allows sharing of health-related data belonging to the respective health-related data type, and (ii) if the user of the ear-wearable device allows sharing of health-related data belonging to the respective health-related data type, a set of one or more users allowed by the user of the ear-wearable device to access the health-related data belonging to the respective health-related data type.

This application claims the benefit of U.S. Provisional Patent Application 62/809,264, filed Feb. 22, 2019, the entire content of which is incorporated by reference.

### TECHNICAL FIELD

This disclosure relates to ear-wearable devices.

### BACKGROUND

Ear-wearable devices are devices designed to be worn on, in, or near one or more of a user's ears. Common types of ear-wearable devices include hearing assistance devices (e.g., “hearing aids” and “hearing instruments”), earbuds, headphones, hearables, cochlear implants, and so on. In some examples, an ear-wearable device may be implanted or osseointegrated into a user. Some ear-wearable devices include additional features beyond just environmental sound-amplification. For example, some modern ear-wearable devices include advanced audio processing for improved device functionality, controlling and programming the devices, and beamforming, and some can even communicate wirelessly with external devices including other ear-wearable devices (e.g., for streaming media).

### SUMMARY

This disclosure describes techniques that enable sharing of health-related data or other types of data based on source data exported by one or more ear-wearable devices. An ear-wearable device may be uniquely situated to generate and collect data related to the health of the user of the ear-wearable device. For instance, many types of health-related data can be determined based on data collected by sensors located near a user's ears. Moreover, many types of ear-wearable devices are already equipped for wireless communication. In examples of this disclosure, an ear-wearable device may export source data to a computing system. The computing system may generate health-related data based on the source data exported by the ear-wearable device. In addition, the computing system may enable a third-party user to access the health-related data that the user of the ear-wearable device authorized to share with the third-party user.

There are a number of challenges associated with the exportation and use of source data from ear-wearable devices. For instance, wirelessly transmitting source data may consume significant amounts of power from a power source of the ear-wearable device. Moreover, a computing system that receives source data and generates health-related data based on the source data should be configured carefully to prevent unauthorized access to the source data and health-related data. This disclosure describes techniques that may reduce power consumption of ear-wearable devices, may reduce the collection and unnecessary dissemination of health-related data by a computing system, and may improve how users of ear-wearable devices interact with computing systems that receive health-related data from ear-wearable devices.

In one example, this disclosure describes a method comprising: receiving, by the computing system, sharing preference data, wherein: the sharing preference data indicate, for each respective health-related data type of a plurality of health-related data types: (i) whether a user of an ear-wearable device allows sharing of health-related data belonging to the respective health-related data type, and (ii) if the user of the ear-wearable device allows sharing of health-related data belonging to the respective health-related data type, a set of one or more users that are allowed by the user of the ear-wearable device to access the health-related data belonging to the respective health-related data type, a set of sharable health-related data types includes each respective health-related data type in the plurality of health-related data types for which the sharing preference data indicate that the user of the ear-wearable device allows sharing of the health-related data belonging to the health-related data types, a set of non-sharable health-related data types includes each respective health-related data type in the plurality of health-related data types for which the sharing preference data indicate that the user of the ear-wearable device does not allow sharing of the health-related data belonging to the health-related data types; for each respective sharable health-related data type of the set of sharable health-related data types, configuring, by the computing system, the ear-wearable device to export source data upon which the health-related data belonging to the respective sharable health-related data type is based; receiving, by the computing system, source data exported by the ear-wearable device; and enabling, by the computing system, a third-party user to access health-related data belonging to a particular sharable health-related data type, wherein: the health-related data belonging to the particular sharable health-related data type is based on the source data exported by the ear-wearable device, and the third-party user is a user allowed by the user of the ear-wearable device to access the health-related data belonging to the particular sharable health-related data type.

In another example, this disclosure describes a computing system comprising: one or more communication units; and one or more processing circuits configured to: receive sharing preference data, wherein: the sharing preference data indicate, for each respective health-related data type of a plurality of health-related data types: (i) whether a user of an ear-wearable device allows sharing of health-related data belonging to the respective health-related data type, and (ii) if the user of the ear-wearable device allows sharing of health-related data belonging to the respective health-related data type, a set of one or more users that are allowed by the user of the ear-wearable device to access the health-related data belonging to the respective health-related data type, a set of sharable health-related data types includes each respective health-related data type in the plurality of health-related data types for which the sharing preference data indicate that the user of the ear-wearable device allows sharing of the health-related data belonging to the health-related data types, a set of non-sharable health-related data types includes each respective health-related data type in the plurality of health-related data types for which the sharing preference data indicate that the user of the ear-wearable device does not allow sharing of the health-related data belonging to the health-related data types; for each respective sharable health-related data type of the set of sharable health-related data types, configure the ear-wearable device to export source data upon which the health-related data belonging to the respective sharable health-related data type is based, wherein the one or more communication units are configured to receive source data exported by the ear-wearable device; and wherein the one or more processing circuits are further configured to enable a third-party user to access health-related data belonging to a particular sharable health-related data type, wherein: the health-related data belonging to the particular sharable health-related data type is based on the source data exported by the ear-wearable device, and the third-party user is a user allowed by the user of the ear-wearable device to access the health-related data belonging to the particular sharable health-related data type.

In another example, this disclosure describes a computing system comprising: means for receiving sharing preference data, wherein: the sharing preference data indicate, for each respective health-related data type of a plurality of health-related data types: (i) whether a user of an ear-wearable device allows sharing of health-related data belonging to the respective health-related data type, and (ii) if the user of the ear-wearable device allows sharing of health-related data belonging to the respective health-related data type, a set of one or more users that are allowed by the user of the ear-wearable device to access the health-related data belonging to the respective health-related data type, a set of sharable health-related data types includes each respective health-related data type in the plurality of health-related data types for which the sharing preference data indicate that the user of the ear-wearable device allows sharing of the health-related data belonging to the health-related data types, a set of non-sharable health-related data types includes each respective health-related data type in the plurality of health-related data types for which the sharing preference data indicate that the user of the ear-wearable device does not allow sharing of the health-related data belonging to the health-related data types; for each respective sharable health-related data type of the set of sharable health-related data types, means for configuring the ear-wearable device to export source data upon which the health-related data belonging to the respective sharable health-related data type is based; means for receiving source data exported by the ear-wearable device; and means for enabling a third-party user to access health-related data belonging to a particular sharable health-related data type, wherein: the health-related data belonging to the particular sharable health-related data type is based on the source data exported by the ear-wearable device, and the third-party user is a user allowed by the user of the ear-wearable device to access the health-related data belonging to the particular sharable health-related data type.

In another example, this disclosure describes a computer-readable data storage medium having instructions stored thereon that, when executed, cause one or more processing circuits of a computing system to: receive sharing preference data, wherein: the sharing preference data indicate, for each respective health-related data type of a plurality of health-related data types: (i) whether a user of an ear-wearable device allows sharing of health-related data belonging to the respective health-related data type, and (ii) if the user of the ear-wearable device allows sharing of health-related data belonging to the respective health-related data type, a set of one or more users that are allowed by the user of the ear-wearable device to access the health-related data belonging to the respective health-related data type, a set of sharable health-related data types includes each respective health-related data type in the plurality of health-related data types for which the sharing preference data indicate that the user of the ear-wearable device allows sharing of the health-related data belonging to the health-related data types, a set of non-sharable health-related data types includes each respective health-related data type in the plurality of health-related data types for which the sharing preference data indicate that the user of the ear-wearable device does not allow sharing of the health-related data belonging to the health-related data types; for each respective sharable health-related data type of the set of sharable health-related data types, configure the ear-wearable device to export source data upon which the health-related data belonging to the respective sharable health-related data type is based; receive source data exported by the ear-wearable device; and enable a third-party user to access health-related data belonging to a particular sharable health-related data type, wherein: the health-related data belonging to the particular sharable health-related data type is based on the source data exported by the ear-wearable device, and the third-party user is a user allowed by the user of the ear-wearable device to access the health-related data belonging to the particular sharable health-related data type.

The details of one or more aspects of the disclosure are set forth in the accompanying drawings and the description below. Other features, objects, and advantages of the techniques described in this disclosure will be apparent from the description, drawings, and claims.

### BRIEF DESCRIPTION OF DRAWINGS

FIG. 1 illustrates an example health monitoring system that includes one or more ear-wearable device(s), a computing system, and a third-party computing device, in accordance with one or more techniques of this disclosure.

FIG. 2 is a flowchart illustrating an example operation of the computing system in accordance with a technique of this disclosure.

FIG. 3 is a conceptual diagram illustrating an example graphical user interface (GUI) that comprises features for health-related data types, in accordance with one or more techniques of this disclosure.

FIG. 4 is a block diagram illustrating example components of an ear-wearable device, in accordance with one or more techniques of this disclosure.

FIG. 5 is a block diagram illustrating example components of a computing device of the computing system, in accordance with one or more techniques of this disclosure.

FIG. 6 is an example GUI for display of a cognitive benefit measure in accordance with one or more techniques of this disclosure.

### DETAILED DESCRIPTION

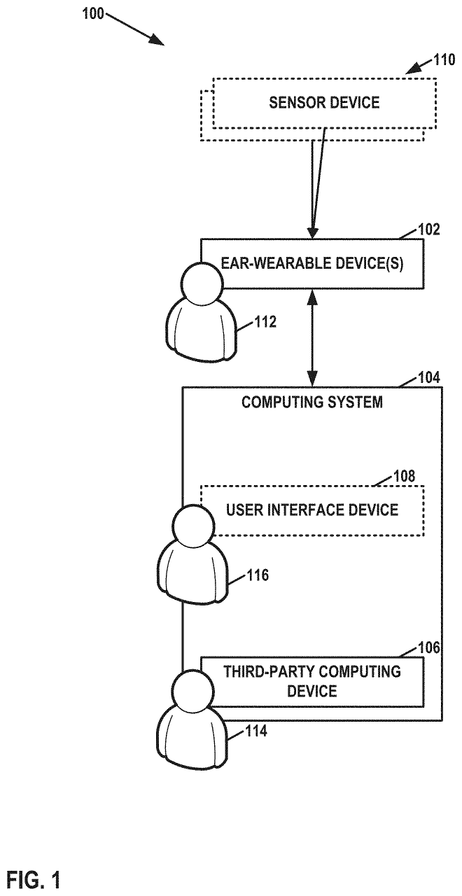

FIG. 1 illustrates an example health monitoring system 100 that includes one or more ear-wearable device(s) 102, a computing system 104, and a third-party computing device 106, in accordance with one or more techniques of this disclosure. In the example of FIG. 1, health monitoring system 100 also comprises a user interface device 108 and one or more sensor devices 110. In other examples, health monitoring system 100 may include more or fewer devices. For instance, in other examples, health monitoring system 100 does not include user interface device 108 and/or sensor devices 110.

Ear-wearable device(s) 102 may comprise one or more of various types of devices that are configured to provide auditory stimuli to user 112 and that are designed for wear and/or implantation at, on, or near an ear of user 112. For instance, in some examples, ear-wearable device(s) 102 may be worn, at least partially, in the ear canal, concha, or behind the ear of user 112. In some examples, ear-wearable device(s) 102 comprise devices that are at least partially implanted into or osseointegrated with the skull of user 112. In some examples, one or more of ear-wearable device(s) 102 is able to provide auditory stimuli to user 112 via a bone conduction pathway. In some instances, such as when user 112 has unilateral hearing loss, user 112 may wear a single ear-wearable device. In other instances, such as when user 112 has bilateral hearing loss, user 112 may wear two ear-wearable devices, with one ear-wearable device for each ear of user 112.

In any of the examples of this disclosure, ear-wearable device(s) 102 may comprise one or more hearing-assistance devices. Hearing assistance devices include devices that help a user hear sounds in the user's environment. Example types of hearing-assistance devices may include hearing aid devices, Personal Sound Amplification Products (PSAPs), cochlear implant systems (which may include cochlear implant magnets, cochlear implant transducers, and cochlear implant processors), and so on. In some examples, ear-wearable device(s) 102 are over-the-counter, direct-to-consumer, or prescription devices. Furthermore, in some examples, ear-wearable device(s) 102 include devices that provide auditory stimuli to user 112 that correspond to artificial sounds or sounds that are not naturally in the user's environment, such as recorded music, computer-generated sounds, or other types of sounds. For instance, ear-wearable device(s) 102 may include so-called “hearables,” earbuds, earphones, or other types of devices. Some types of ear-wearable devices provide auditory stimuli to the user corresponding to sounds from the user's environmental and also artificial sounds.

In some examples, one or more of ear-wearable device(s) 102 includes a housing or shell that is designed to be worn in the ear for both aesthetic and functional reasons and encloses the electronic components of the hearing instrument. Such ear-wearable devices may be referred to as in-the-ear (ITE), in-the-canal (ITC), completely-in-the-canal (CIC), or invisible-in-the-canal (IIC) devices. In some examples, one or more of ear-wearable device(s) 102 may be behind-the-ear (BTE) devices, which include a housing worn behind the ear that contains all of the electronic components of the hearing instrument, including the receiver (i.e., the speaker). The receiver conducts sound to an earbud inside the ear via an audio tube. In some examples, one or more of ear-wearable device(s) 102 may be receiver-in-canal (RIC) hearing-assistance devices, which include a housing worn behind the ear that contains electronic components and a housing worn in the ear canal that contains the receiver.

Computing system 104 comprises one or more computing devices. For instance, computing system 104 comprises one or more mobile devices, server devices, personal computer devices, handheld devices, wireless access points, smart speaker devices, smart televisions, medical alarm devices, smart key fobs, smartwatches, smartphones, motion or presence sensor devices, smart displays, screen-enhanced smart speakers, wireless routers, wireless communication hubs, prosthetic devices, mobility devices, special-purpose devices, accessory devices, and so on. Accessory devices may include devices that are configured for use with ear-wearable device(s) 102. Example types of accessory devices may include charging cases for ear-wearable device(s) 102, storage cases for ear-wearable device(s) 102, media streamer devices, phone streamer devices, external microphone devices, remote controls for ear-wearable device(s) 102, and other types of devices specifically designed for use with ear-wearable device(s) 102. Actions described in this disclosure as being performed by computing system 104 may be performed by one or more of the computing devices of computing system 104.

In the example of FIG. 1, computing system 104 includes a third-party computing device 106. Third-party computing device 106 is a computing device associated with a third-party user 114. Third-party user 114 may be a user other than user 112 and a manufacturer of ear-wearable device(s) 102. For instance, third-party user 114 may be a person (e.g., a family member, a loved one, a physician, or a caregiver), an organization, a group of persons and/or organizations, who is interested in health-related data of user 112.

In some examples of this disclosure, third-party user 114 is not necessarily a user of any ear-wearable devices. In other examples, third-party user 114 is an ear-wearable device user. In such examples, third-party user 114 may or may not be interested in the health-related data of a specific user or users, such as user 112. In some examples where third-party user 114 is interested in the health-related data of a specific user or users, computing system 104 may enable third-party user 114 to explicitly connect, in a similar manner to social networking services such as Facebook or Twitter, with a specific user or users in order to share health-related data for the purpose of competition or motivation. Furthermore, in some examples where third-party user 114 is a user of ear-wearable devices and is not interested in the health-related data of a specific user, third-party user 114 may want to compare their own health-related data to, and share their data with, anonymized groups of their peer ear-wearable device users, such as user 112. For the purpose of some forms of competition, third-party user 114 may be paired with a single person, a small group of persons, or a wellness community.

In some examples, third-party user 114 is an organization. For instance, third-party user 114 is an emergency services dispatching organization. Thus, third-party user 114 may be able to review health-related data regarding user 112 in order to alert first responders, hospital personnel, or other persons of one or more health conditions of user 112 during an emergency. In other examples, third-party user 114 is a medical company or an insurance company.

Third-party computing device 106 may comprise various types of computing devices, such as a personal computer, smartphone, tablet computer, special-purpose computing device, or another type of computing device. In any of the examples described in this disclosure, third-party computing device 106 may be associated with third-party user 114 in the sense that third-party user 114 has registered third-party computing device 106 as being associated with third-party user 114 (e.g., by logging into a service of computing system 104 using third-party computing device 106).

Additionally, as shown in the example of FIG. 1, computing system 104 may include a user interface device 108. User interface device 108 is a computing device that is configured to provide a user interface configured to receive indications of user input. User 112 may provide the indications of user input to the interface. Furthermore, a user 116 may provide the indications of user input to the interface on behalf of user 112. User 116 may be an audiologist, a caregiver, a family member, a representative of a health monitoring service, third-party user 114, or another type of person.

User interface device 108 may comprise one of various types of computing devices, such as a personal computer, a smartphone, a tablet computer, a wearable device (e.g., a smartwatch device, bracelet device), a handheld computing device, a special-purpose computing device, or another type of computing device. Although shown in the example of FIG. 1 as being part of computing system 104, user interface device 108 may, in other examples, be one or more of ear-wearable device(s) 102. Furthermore, user interface device 108 and third-party computing device 106 may be the same device. Accordingly, it should be understood that references in this disclosure to user interface device 108 may apply instead to one of ear-wearable device(s) 102, a computing device of computing system 104, third-party computing device 106, or another computing device. Computing system 104 may include computing devices in addition to third-party computing device 106 and user interface device 108.

The user interface provided by user interface device 108 may comprise a graphical user interface (GUI), a voice interface, a text-based interface, a camera-based user interface, or another type of user interface. In some examples where the user interface provided by user interface device 108 comprises a GUI, a web browser application may present the GUI as a webpage. Furthermore, in some examples where the user interface provided by user interface device 108 is a GUI, a special-purpose application installed on user interface device 108 may present the GUI. In some examples where the interface provided by user interface device 108 is a text-based interface, user interface device 108 may receive and send text messages (e.g., short message service (SMS) messages, instant messages, chat messages, etc.). In some examples where the interface provided by user interface device 108 is a voice interface, user interface device 108 may generate audible questions and receive spoken responses. In some examples, one or more of ear-wearable device(s) 102, computing devices of computing system 104, third-party computing device 106, and/or user interface device 108 may be equipped with a virtual personal assistant, such as ALEXA™ from Amazon.com, Inc. or GOOGLE ASSISTANT™ from Google LLC.

Sensor devices 110 may include devices having one or more sensors that are configured to gather information about user 112. In any example of this disclosure, one or more of sensor devices 110 includes a body-worn device, such as a smartwatch, smart glasses, an implantable medical device, a belt-worn device, an adhesive body-worn patch, a Holter monitor, and so on. Example types of sensors may include electrocardiogram (EKG) sensors, photoplethysmogram (PPG) sensors, heart rate sensors, body temperature sensors, inertial measurement units (IMUs), accelerometers, gyroscopes, electroencephalogram (EEG) sensors, magnetometers, image sensors, cameras, respiration sensors, pulse oximetry sensors, blood pressure sensors, eye movement sensors, eye-tracking sensors, microphones, pressure sensors, and so on.

Ear-wearable device(s) 102 may be configured to communicate with each other and sensor devices 110. For instance, in any of the examples of this disclosure, ear-wearable device(s) 102 may communicate with each other and/or with sensor devices 110 using one or more wirelessly communication technologies. Example types of wireless communication technology include Near-Field Magnetic Induction (NFMI) technology, a 900 MHz technology, a BLUETOOTH™ technology, a WI-FI™ technology, audible sound signals, ultrasonic communication technology, infrared communication technology, an inductive communication technology, or another type of communication that does not rely on wires to transmit signals between devices. In some examples, ear-wearable device(s) 102, computing devices of computing system 104, and sensor devices 110 may use a 2.4 GHz frequency band for wireless communication. In some examples, sensor devices 110 communicate with ear-wearable device(s) 102 and not with any other devices, such as computing devices of computing system 104. In any of the examples of this disclosure, one or more of ear-wearable device(s) 102 may communicate with each other or with sensor devices 110 via non-wireless communication links, such as via one or more cables, direct electrical contacts, and so on.

Sensors of ear-wearable device(s) 102 and/or sensor devices 110 may generate signals that may be used to monitor user 112 for signs of various medical conditions, levels of physical activity, levels of social activity, levels of cognitive health, and other health-related data. For example, the sensors may generate signals that may be used to monitor user 112 for signs that user 112 has fallen. In other examples, the sensors may generate signals that may be used to monitor the heart rate of user 112, generate an electrocardiogram of user 112, measure a respiration rate of user 112, measure a blood pressure of user 112, and measure the blood glucose of user 112. Furthermore, in some examples, the sensors may sense tremors that may be associated with epilepsy, Parkinson's disease, or other conditions. In some examples, one or more of the sensors may measure snoring or signals indicative of the quality of sleep of user 112.

Ear-wearable device(s) 102 are configured to communicate with computing system 104. In any of the examples of this disclosure, ear-wearable device(s) 102 may use one or more wireless communication technologies to communicate with computing system 104. For example, ear-wearable device 102 and computing system 104 may communicate wirelessly using a BLUETOOTH™ technology, a WIFI™ technology, an ultrasonic communication technology, or another type of wireless communication technology. Furthermore, in any of the examples of this disclosure, ear-wearable device(s) 102 may be configured to use one or more non-wireless technologies to communicate with computing system 104.

As previously mentioned, sensors of ear-wearable device(s) 102 and/or sensor devices 110 generate signals that may be used to monitor user 112 for signs of various medical conditions, physical activity, cognitive and social wellness, and other health-related factors. In any of the examples of this disclosure, ear-wearable device(s) 102 may generate data based on signals generated by the sensors of ear-wearable device(s) 102 and/or the sensors of sensor devices 110. This disclosure may refer to this data as source data because this data may be used as a source for generating health-related data or may itself be health-related data. Ear-wearable device(s) 102 may generate source data in various ways. For instance, in any of the examples of this disclosure, the source data may include data generated by sensors. Furthermore, in any of the examples of this disclosure, ear-wearable device(s) 102 may apply various functions or algorithms to data generated by sensors to generate the source data.

Ear-wearable device(s) 102 are configured to selectively export particular types of source data to computing system 104. For instance, ear-wearable device(s) 102 may be configured to use one or more wireless or non-wireless communication technologies to send source data to computing system 104. Thus, computing system 104 may receive source data exported by ear-wearable device(s) 102.

Computing system 104 may enable third-party user 114 to access health-related data belonging to a sharable health-related data type, provided that third-party user 114 is a user (e.g., person, organization, group of persons and/or organizations) allowed by user 112 to access the health-related data belonging to the sharable health-related data type. The health-related data belonging to the sharable health-related data type is based at least in part on the source data exported by ear-wearable device(s) 102. In some examples, the health-related data belonging to the sharable health-related data type is also based on source data obtained by computing system 104 from other sources.

In any of the examples of this disclosure, computing system 104 may generate the health-related data based on the source data exported by ear-wearable device(s) 102 and, in some examples, source data obtained from other sources, such as accessory devices, sensor devices 110, or other types of devices. For instance, in any of the examples of this disclosure, computing system 104 may apply one or more algorithms that transform the source data into the health-related data. Such algorithms may be specific to the types of health-related data being generated. Thus, computing system 104 may use a first algorithm to generate health-related data describing the heart rate of user 112, a second algorithm to generate health-related data describing the cognitive wellness of user 112, and so on. Example types of algorithms usable in generating health-related data may include neural network algorithms, algorithms applying mathematical transformations or filters, and so on. Furthermore, in any of the examples of this disclosure, the health-related data may directly include the source data exported by ear-wearable device(s) 102. For particular health-related data types, the source data may include the health-related data belonging to the particular health-related data types, and hence ear-wearable device(s) 102 may generate the health-related data belonging to the particular health-related data types.

Ear-wearable device(s) 102 may be configured to generate and export one or more of various types of source data. In other words, ear-wearable device(s) 102 may be configured to generate and export source data upon which health-related data belonging to a plurality of health-related data types are based. Example types of health-related data types may include data indicating the heart rate of user 112, heart rate recovery data, electrocardiogram (ECG) data, electroencephalogram (EEG) data, galvanic skin response data, blood glucose data, blood pressure data, heart rate variability data, blood oxygenation data, body temperature data, environmental temperature data, environmental humidity data, body hydration level data, physical activity data, social wellness data, data regarding balance checks, fall detection data, fall risk data, presence of tinnitus, geographic location data, and other types of data that may be used for determining aspects of the mental and/or physical health condition of user 112.

Because some types of health-related data are of a very personal nature, user 112 may not wish to share some types of health-related data. Moreover, even if user 112 does allow sharing of a particular health-related data type, user 112 may want to limit distribution of health-related data belonging to the particular health-related data type to particular people, particular organizations, or particular types of people. That is, different ear-wearable device users may have different preferences about what health-related data types they are willing to share and with whom. At the same time, operators of computing system 104 may want to minimize the amount of health-related data stored by computing system 104 to minimize storage requirements, to minimize the amount of data that could potentially be obtained without authorization, and for other reasons.

Conventionally, users of ear-wearable devices that have embedded sensors or that communicate with sensors do not have a means by which to control sharing of health-related data based on information from such sensors selectively with professionals, physicians, relatives, caregivers, other hearing aid users, or other types of third-party users. In the absence of being able to share health-related data selectively, users of ear-wearable devices may opt to not share any health-related data. As a result, potentially life-improving intervention by third-party users may be unavailable for users of ear-wearable devices, especially in the instance where users of ear-wearable devices are unable to manually solicit such intervention. For example, a heart rate sensor in an ear-wearable device may detect an abnormally high heart rate, but conventionally does not provide any means for third-party users to be alerted of such a health condition, which might require scheduling a medical appointment for diagnosis of an underlying health condition or contacting emergency services if merited.

In addition, there is data to show improved outcomes for wellness when users share and are accountable to others for reaching goals. Sharing data as part of a similar demographic group or competitive venture can increase success in wellness goals or faster acclimatization to wellness devices like hearing aids, fitness sensors, or heart-rate monitors. Furthermore, conventional systems do not enable users of ear-wearable devices to share health-related data for the purpose of competition with other users of ear-wearable devices. Additionally, conventional systems do not enable selective sharing of health-related data with third-party users for the purpose of creating awareness of a health condition of an ear-wearable device user (e.g., user 114). The third-party users may include family members, loved ones, or caregivers of ear-wearable device users who are not directly responsible for the medical care of the user of the ear-wearable devices, but who are invested in the wellbeing of the user of the ear-wearable devices.

Hence, in accordance with the techniques of this disclosure, a user interface provided by user interface device 108 may comprise separate features for each respective health-related data type of a plurality of health-related data types. For each respective health-related data type of the plurality of health-related data types, the features for the respective health-related data type enable an interface user (e.g., user 112 or user 116) to specify sharing preferences for the respective health-related data type of user 112 of ear-wearable device(s) 102. In any of the examples of this disclosure, the sharing preference data may indicate, for each respective health-related data type of the plurality of health-related data types:
(i) whether user 112 of the ear-wearable device(s) 102 allows sharing of health-related data belonging to the respective health-related data type to be shared, and
(ii) if user 112 of ear-wearable device(s) 102 allows sharing of health-related data belonging to the respective health-related data type, a set of one or more users allowed by user 112 of ear-wearable device(s) 102 to access the health-related data belonging to the respective health-related data type.

In some examples, computing system 104 may store data specifying a list of users allowed to access health-related data belonging to a health-related data type. In some such examples, the existence of this list alone may indicate that user 112 allows sharing of the health-related data belonging to the health-related data type. In other examples, computing system 104 may store separate data indicating (i) and (ii). In some examples, in addition to the plurality of health-related data types for which sharing preference data is present, there may be one or more health-related data types that are related to the health of user 112 that may be shared with one or more third-party users without receiving sharing preference data indicating explicit selection from user 112 (e.g., for regulatory or insurance purposes).

The health-related data types may include a set of sharable health-related data types and a set of non-sharable health related data types. The set of sharable health-related data types includes each respective health-related data type in the plurality of health-related data types for which the sharing preference data indicate that user 112 of ear-wearable device(s) 102 allows sharing of the health-related data belonging to the health-related data types. The set of non-sharable health-related data types includes each respective health-related data type in the plurality of health-related data types for which the sharing preference data indicate that user 112 of ear-wearable device(s) 102 does not allow sharing of the health-related data belonging to the health-related data types. In some examples, computing system 104 may determine, based on the sharing preference data, which of the health-related data types are sharable health-related data types and which of the health-related data types are non-sharable health-related data types (e.g., by scanning through a predetermined list of health-related data type and marking the health-related data types as a sharable health-related data type if the sharing preference data indicates a health-related data type is a sharable health-related data type and indicating a health-related data type is a non-sharable health-related data type if the sharing preference data indicates the health-related data type is a non-sharable health-related data type.)

In some examples, the health-related data types may also include a set of sharable-but-anonymized health-related data types. The set of sharable-but-anonymized health-related data types may include each respective health-related data type in the plurality of health-related data types for which the sharing preference data indicate that user 112 of ear-wearable device(s) 102 allows sharing of health-related data belonging to the health-related data types but only in anonymized form. Computing system 104 may anonymize the health-related data by removing personally-identifying information from the health-related data and/or source data. Examples of this disclosure that apply with respect to sharable health-related data types may apply with respect to sharable-but-anonymized health-related data types. For instance, like sharable health-related data types, sharing preference data may indicate a set of one or more third-party users who are allowed by user 112 to access sharable-but-anonymized health-related data belonging to sharable-but-anonymized health-related data types. In some examples, a user interface (e.g., a user interface presented by user interface device 108) may allow indication of sharable, non-sharable, or sharable-but-anonymized health-related data type options for a set of health-related data types.

User 112 (or another user with authorization from user 112) may update the sharing preference data. In this way, for each user to whom user 112 grants permission to access their health-related data, user 112 can select a subset of their total health-related data set to share. This subset can be different for each user with whom 112 shares data and can be modified upon a granted request from third-party user 114 or by initiation of user 112.

As noted above, the user interface provided by user interface device 108 may comprise separate features for each respective health-related data type of a plurality of health-related data types. The features of the user interface provided by user interface device 108 for the health-related data types may have various forms. For instance, in examples where the user interface comprises a graphical user interface, the features may include graphical elements, such as text boxes, drop boxes, radio buttons, sliders, toggle boxes, and so on. In examples where the user interface comprises a voice interface, the features may include verbal questions that request verbal responses.

Based on the sharing preference data, computing system 104 may, for each respective sharable health-related data type of the set of sharable health-related data types, configure ear-wearable device(s) 102 to export source data upon which health-related data belonging to the respective sharable health-related data type is based. Moreover, computing system 104 may configure ear-wearable device(s) 102 not to export embargoed source data. The embargoed source data is source data upon which health-related data belonging to one or more health-related data types of the plurality of health-related data type are based, but which is not to be exported by ear-wearable device(s) 102. Thus, for each respective sharable health-related data type, the health-related data belonging to the respective sharable health-related data type is not based on the embargoed source data. Moreover, health-related data belonging to one or more of the non-sharable health-related data types may be based on the embargoed source data. For instance, computing system 104 may not be able to generate non-sharable health-related data because computing system 104 the embargoed source data is not exported to computing system 104. Computing system 104 may determine which types of source data are identified as embargoed source data by checking a list of types of source data needed to generate or otherwise provide sharable health-related data types and marking the remaining types of source data as embargoed source data.

In this way, health monitoring system 100 may, with permission of user 112, provide third-party users (e.g., professionals, physicians, relatives, demographic peers, caregivers/other hearing aid users) access to health-related data, such as data describing one or more aspects of the health of user 112, the wellness of user 112, physiological sensor data that represent measurements of user 112, and other types of health-related data to provide insight into health conditions and/or health goals of user 112. This may allow the third-party users to obtain information regarding a physical or emotional state of user 112 in order to intervene on behalf of user 112 if necessary, to monitor and stay informed of the wellbeing of user 112, or to compare their own derived health-related data to that of user 112, e.g., for the purpose of competition. At the same time, by configuring ear-wearable device(s) 102 not to export the embargoed source data, the energy consumption of ear-wearable device(s) 102 may be reduced, storage requirements of computing system 104 may be reduced, unnecessary collection and dissemination of health-related data (with inherent increased risk of privacy breach) may be reduced, and bandwidth may be conserved because potentially less data may be transferred.

As mentioned above, computing system 104 may enable third-party user 114 to access health-related data belonging to particular sharable health-related data types. Computing system 104 may enable third-party user 114 to access the health-related data in one or more of various ways. For instance, computing system 104 may enable third-party user 114 to access the health-related data via a user interface of third-party computing device 106. The user interface provided by third-party computing device 106 may comprise a GUI, a voice interface, a text-based interface, a camera-based user interface, or another type of user interface. In some examples where the user interface provided by third-party computing device 106 comprises a GUI, a web browser application may present the GUI as a webpage. Furthermore, in some examples where the user interface provided by third-party computing device 106 is a GUI, a special-purpose application installed on third-party computing device 106 may present the GUI. In some examples where the interface provided by third-party computing device 106 is a text-based interface, third-party computing device 106 may receive and send text messages (e.g., short message service (SMS) messages, instant messages, chat messages, etc.).

In some examples where the interface provided by third-party computing device 106 is a voice interface, third-party computing device 106 may generate audible questions and receive spoken responses, and vice versa. For example, if the name of user 112 is “Chris,” third-party user 114 may be able to ask, “What is Chris' current step count?” or “What is Chris' average heart rate today?”

In some examples, computing system 104 may receive a request from third-party user 114 via a user interface (e.g., a GUI or voice interface) for performance of a self-check on ear-wearable device(s) 102 to determine whether there are problems with ear-wearable device(s) 102. In some examples, computing system 104 may implement an AI system that identifies a problem with ear-wearable device(s) 102 based on self-check data received from ear-wearable device(s) 102. For instance, a neural network may be trained to identify known types of problems with ear-wearable device(s) 102 based on self-check data generated by ear-wearable device(s) 102 or other sources.

In some examples where third-party user 114 accesses health-related data via a GUI of third-party computing device 106, third-party computing device 106 may output a dashboard for display. The dashboard may provide information regarding real-time sensed health parameters, a health scoring system, a list of discrete sensor-measured events outside of a normal physiological range, or other health-related information.

In some examples, computing system 104 is configured to provide notifications to third-party user 114. The notification may alert third-party user 114 when user 112 experiences a predefined health-related condition. In some examples, the notifications may take the form of audible messages, video messages, text messages (e.g., short message service (SMS) messages, instant messages, chat messages, email messages, etc.), tactile or haptic signals, and so on. The health-related conditions may be mental, physical, or a combination thereof. In some examples, user 112, third-party user 114, or another user defines the health-related conditions that cause computing system 104 to generate notifications.

In some examples, health monitoring system 100 implements an AI system that determines whether to generate a notification. For instance, ear-wearable device(s) 102 and/or computing system 104 may implement the AI system that determines whether to generate the notification. In some examples, the AI system may comprise an artificial neural network that is trained to generated notifications. For instance, the AI system may be trained to identify conditions under which to generate particular types of notifications based on various types of health-related data or data derived therefrom, such as a deviation from or correlation with previously-observed data.

In any of the examples of this disclosure, computing system 104 may enable user 112 to access health-related data via a user interface of a computing device associated with user 112. The computing device associated with user 112 may present the health-related data (e.g., in visible, tactile, and/or audible form) to user 112. The computing device associated with user 112 may be user interface device 108, ear-wearable device(s) 102, a mobile device, a personal computer, an accessory device, or another type of computing device. The computing device associated with user 112 may be associated with user 112 in the sense that user 112 has logged-in to the computing device, computing system 104 otherwise stores data that records user 112 as being a user of the computing device or associated in another way. The user interface of the computing device associated with user 112 may be any of the types of user interface described elsewhere in this disclosure with respect to user interface types of third-party computing device 106 or user interface device 108. The health-related data accessible to user 112 may be the same or different from health-related data accessible to third-party user 114. In some examples, the health-related data accessible to user 112 through the computing device associated with user 112 enables user 112 to compare and/or compete with other users with respect to health-related data.

In addition to accessing and viewing their health-related data, the computing device associated with user 112 may, in any of the examples of this disclosure, also receive indications of user input specifying comments on the health-related data. For instance, user 112 may provide the comments in order to provide contextual information regarding health-related data. User 112 may provide the comments in order to ask questions of third-party user 114 in order to solicit feedback about, or interpretation of, the health-related data. For instance, in an example where user 114 is a physician of user 112, user 112 may ask the physician if user 112 should be concerned about a particular measurement, set of measurements, or trends across a series of measurements included in the health-related data of user 112.

Thus, in any of the examples of this disclosure, computing system 104 may receive comment data generated by user 112 of ear-wearable device(s) 102. The comment data may be associated with one or more sharable health-related data types. Furthermore, for each respective sharable health-related data type of the set of sharable health-related data types, computing system 104 may determine whether there is comment data associated with the respective sharable health-related data type. In response to determining that there is comment data associated with the respective sharable health-related data type, computing system 104 may generate health-related data belonging to the respective sharable health related data type based on the source data exported by the ear-wearable device and include, in the health-related data belonging to the respective sharable health-related data type, the comment data associated with the respective sharable health-related data type.

Furthermore, in any of the examples of this disclosure, in addition to accessing and viewing health-related data regarding user 112, third-party user 114 also may comment upon the health-related data in order to provide feedback, encouragement, or guidance, to inquire about further details of the data or the context surrounding the health-related data, or for other purposes. Third-party user 114 may also set goals for user 112 in an effort to motivate user 112 to improve one or more aspects of the health of user 112, to motivate user 112 to wear ear-wearable device(s) 102 for longer periods of time, or to achieve some objective related to the health-related data. Thus, in any of the examples of this disclosure, computing system 104 may receive message data comprising a message from third-party user 114 to user 112 of ear-wearable device(s) 102 and computing system 104 may send the message data to a computing device associated with user 112 of ear-wearable device(s) 102.

In any of the examples of this disclosure, third-party user 114 may initiate reminders to user 112 via computing system 104 in order to encourage user 112 to complete some behavior in a timely manner. For instance, in an example where third-party user 114 is a physician and user 112 is a patient, the physician may wish to remind the patient to take a particular medication with breakfast and lunch, or to check their blood pressure each morning. In examples where third-party user 114 is able to initiate reminders, third-party user 114 may use an interface of third-party computing device 106 to instruct computing system 104 to send the reminder to a computing device associated with user 112 (e.g., one or more of ear-wearable device(s) 102, a smartphone of user 112, or another device associated with user 112). In some examples, computing system 104 may send reminders to user 112 in the form of email messages, text messages (e.g., SMS messages, instant messages), application-based notifications, telephone calls, voicemail messages, and so on.

In accordance with any of the examples of this disclosure, user 112 may grant third-party user 114 permission to initiate measurements of one or more types of health-related data, such as heart rate or temperature. This may be particularly useful for ear-wearable device users who are not able or not inclined to carefully monitor their health, even though health monitoring may have significant benefits to such ear-wearable device users. For example, third-party user 114 may want to invest in the health monitoring capabilities of ear-wearable device(s) 102 for user 112, knowing that user 112 is less interested in, or less able to, monitor their own health. This may provide third-party user 114 with a way to collect updated health and wellness data provided by ear-wearable device(s) 102, with the permission of user 112, but without requiring user 112 to initiate the measurements themselves.

Thus, in any of the examples of this disclosure, computing system 104 may receive first request data from a computing device associated with third-party user 114 (e.g., third-party computing device 106). The first request data comprises a request for updated health-related data belonging to the particular sharable health-related data type. In response to receiving the first request data, computing system 104 may send second request data to ear-wearable device(s) 102. Computing system 104 may then receive updated source data from ear-wearable device(s) 102 in response to the second request data. Computing system 104 may generate the updated health-related data based on the updated source data. Furthermore, computing system 104 may enable third-party user 114 to access the updated health-related data. Computing system 104 may enable third-party user 114 to access the updated health-related data in any of the ways described elsewhere in this disclosure.

In any of the examples of this disclosure, third-party user 114 may set up alerts within computing system 104 so that computing system 104 notifies third-party user 114 when particular conditions occur. For example, third-party user 114 may configure computing system 104 to generate alerts whenever new health-related data related to user 112 becomes available to third-party user 114. In some examples, third-party user 114 may configure computing system 104 to generate an alert if a particular measurement of user 112 has not been made for a particular amount of time. In some examples, third-party user 114 may configure computing system 104 to generate an alert if the result of a particular measurement of user 112 is outside of a range of desirable values. In such examples, the range of desirable values may be set by user 112, third-party user 114, or another person or organization (e.g., a medical provider of user 112).

In any of the examples of this disclosure, user 112 may provide third-party users selective access to one or more health-related data types using permissions, text messages or email, a shared key, or other sources of communication and authentication. For instance, user 112 may send health-related data to a third-party user using text messages or email; may allow the third-party user to access stored health-related data by granting permissions, sharing an encryption key, or otherwise; or may otherwise enable sharing with the third-party user.

Furthermore, in any of the examples of this disclosure, third-party user 114 may use third-party computing device 106 or another computing device to input a search request for a specific user (e.g., by entering the user's name or other identifying information) and then sending to computing system 104 an electronic request for access to the information. Computing system 104 may provide the electronic request to a computing device associated with user 112. Upon receiving the request, user 112 may provide input to the computing device associated with user 112 indicating that user 112 accepted or rejected the request.

Furthermore, in any of the examples of this disclosure, communication between third-party user 114 and user 112 may be asynchronous in that both parties do not have to be connected to the shared data at the same time in order for communication to take place. However, accessibility of health-related data may be synchronous in that third-party user 114 may able to access to new health-related data as soon as they are available from computing system 104.

In one or more examples, computing system 104 may receive sharing preference data that comprises separate features for each respective health-related data type of a plurality of health-related data types. Examples of health-related data types may include:
Heart rate data. The heart rate data may include one or more of real-time measurements of the heart rate of user 112, measurements of the heart rate of user 112 generated at defined monitoring intervals, measurements of the heart rate of user 112 generate for discrete events (e.g., a resting heart rate of user 112, a walking heart rate of user 112, a running heart rate of user 112, a peak heart rate of user 112, and so on), or other types of data regarding the heart rate of user 112.
Heart rate recovery data that provides information regarding how quickly the heart rate of user 112 returns to a resting heart rate. For example, ear-wearable device(s) 102 may determine a heart rate of user 112 at the time that the user stops exercising and the heart rate of user 112 at a given time later (e.g., 2 minutes). In this example, ear-wearable device(s) 102 may determine the user 112 has stopped exercising based on signals from an IMU or other sensor.
Electrocardiogram (ECG) data. The ECG data may include one or more of real-time ECG measurements of user 112, ECG measurements generated at defined monitoring intervals, ECG measurements generated during discrete events, or other types of ECG measurements of user 112.
Electroencephalogram (EEG) data. The EEG data may include one or more of real-time EEG measurements of user 112, EEG measurements generated at defined monitoring intervals, EEG measurements generated during discrete events, or other types of EEG measurements of user 112. The EEG may enable computing system 104 to generate monitor stress/arousal levels of user 112. For instance, ear-wearable device(s) 102 or computing system 104 may estimate a stress or arousal level of user 112 based on a combination of alpha waves of the EEG, beta waves of the EEG, a respiration rate, a body temperature, a heart rate, and/or other factors.
Galvanic skin response data. The galvanic skin response data may include one or more of real-time galvanic skin response measurements of user 112, galvanic skin response measurements generated at defined monitoring intervals, galvanic skin response measurements generated during discrete events, or other types of data regarding the galvanic skin response of user 112.
Blood glucose data. The blood glucose data may include one or more of real-time blood glucose measurements, blood glucose measurements generated at defined monitoring intervals, blood glucose measurements generated during discrete events, or other types of data regarding blood glucose levels of user 112.
Blood pressure data. The blood pressure data may include one or more of real-time blood pressure measurements, blood pressure measurements generated at defined monitoring intervals, blood pressure measurements generated during discrete events, or other types of data regarding the blood pressure of user 112.
Heart rate variability data. The heart rate variability data may include one or more of real-time data regarding variability of the heart rate of user 112, data regarding the variability of the heart rate of user 112 as measured during defined monitoring intervals, data regarding the variability of the heart rate of user 112 as measured during discrete events, or other data regarding the heart rate variability of user 112.
Blood oxygenation data. The blood oxygenation data may include one or more of real-time blood oxygenation measurements of user 112, blood oxygenation measurements of user 112 generated at defined monitoring intervals, blood oxygenation measurements of user 112 generated during discrete events, or other data regarding the blood oxygenation of user 112.
Body temperature data. The body temperature data may include one or more of real-time body temperature measurements, body temperature measurements generated at defined monitoring intervals, body temperature measurements generated during discrete events, or other data regarding the body temperature of user 112. In some examples, computing system 104 may generate low and high body temperature alerts based on the body temperature data.
Body hydration level data. The body hydration level data may include real-time body hydration level measurements, body hydration level measurements generated according to defined monitoring intervals, body hydration level measurements generated during discrete events, or other types of data regarding the body hydration level of user 112.
Cognitive fitness data. The cognitive fitness data may be updated in real time, according to defined monitoring intervals, during discrete events, as a daily score, or according to other regimes.
Physical activity data. The physical activity data may include step count, activity type (at rest, walking, running, biking, aerobics), and so on. In some examples, the physical activity data may include heart rate, breathing rate, temperature, hydration, and/or other metrics as function of activity.
Thrive score data. The thrive score data may indicate an overall level of wellness of user 112. The thrive score data may be updated in real time, according to defined monitoring intervals, during discrete events, or according to other regimes.
Cognitive benefit measure. The cognitive benefit measure may also be referred to as a “brain score”. The brain score data (and components thereof) may be updated in real time, according to defined monitoring intervals, during discrete events, or according to other regimes.
Body fitness measure, such as a body score (and components). The body score data may be updated in real time, according to defined monitoring intervals, during discrete events, or according to other regimes.
Time duration for a selection of physiological parameters. For instance, this type of health-related data may indicate the number of occurrences of an event (e.g., an arrythmia, body temperatures over particular thresholds, etc.) that occurred within particular time periods.
Balance training, fall risk, fall detection data.
Respiratory symptom data, such as breathing rate, wheezing, and so on.
Prescription compliance, medicine reminders, alerts for missed doses
Meditation data. This may include mindfulness training, relaxation techniques.
Cognitive enhancement training data. This may include data regarding the participation of user 112 in brain training games.
Tinnitus-related data. Tinnitus-related data may include one or more of use of tinnitus masker, performance of cognitive behavioral therapy for treatment of tinnitus, or other information related to tinnitus.
Appetite data indicating a perceived appetite of user 112.
Affective state data.

In addition to enabling user 112 to selectively share health-related data, computing system 104 may enable user 112 to selectively share other types of data. User 112 may be able to selectively share such other types of data in a manner similar to the health-related data described elsewhere in this disclosure. Computing system 104 may use these other types of data to generate particular types of health-related data. For example, computing system 104 may enable user 112 to selectively share various types of data such as:
Geolocation data. The geolocation data may include data related to the location of user 112. The geolocation data may be based on satellite positioning services, such as the Global Positioning Service (GPS). The geolocation data may be based on acceleration/velocity data and a map system. The geolocation data may include real-time location data, location data collected at defined monitoring intervals, location data collected during discrete events, or other data regarding the geographic location of user 112.
Location type data. The location type data may indicate, for example, whether user 112 is at home, at work, at gym, while driving a vehicle (indicated by accelerometer, GPS, velocity data), or at other types of locations.
Environmental temperature data. The environmental temperature data may include real-time environmental temperature measurements, environmental temperature measurements generated according to defined monitoring intervals, environmental temperature measurements generated during discrete events, or other types of data regarding the environmental temperature in the environment of user 112.
Environmental humidity data. The environmental humidity data may include real-time environmental humidity measurements, environmental humidity measurements generated according to defined monitoring intervals, environmental humidity measurements generated during discrete events, or other types of data regarding the environmental humidity in the environment of user 112.
User profile data
Overall use of ear-wearable device(s) 102, which may indicate whether the ear-wearable devices are being worn?)
Ear-wearable device self-check data. The ear-wearable device self-check data may include data regarding the performance of ear-wearable device(s) 102. In some examples, computing system 104 may enable user 112 and/or third-party user 114 to access a dashboard that reports on the condition of one or more components of ear-wearable device(s) 102, such as whether a microphone working, whether the IMU working, and so on. Failure of one or more components of ear-wearable devices is a common reason why users stop wearing ear-wearable devices. Accordingly, the ear-wearable device self-check data may help third-party user 114 determine why user 112 might not be wearing ear-wearable device(s) 102.

In some examples, health-related data, non-health-related data, and source data may be timestamped. For instance, computing system 104 may associate a time of day (e.g., morning, afternoon, evening) with these types of data. In some examples, the timestamps may enable computing system 104 to track trends in the data, filter the data, or perform other types of time-based analysis on the data.

As mentioned elsewhere in this disclosure, the health-related data may include a cognitive benefit measure. The cognitive benefit measure may be a measure of the social and emotional wellbeing of user 112. Furthermore, in some examples, the health-related data may include a body fitness measure that may be an indication of physical activity in which user 112 of ear-wearable device(s) 102 engages. Computing system 104 may calculate a cognitive benefit measure, body fitness measure, and thrive score in any one of various ways. For instance, computing system 104 may calculate the cognitive benefit measure, body fitness measure, and thrive score in accordance with any of the examples set forth in U.S. patent application Ser. No. 16/110,996, filed Aug. 23, 2018.

Computing system 104 may store source data, health-related data, and/or other types of data. For instance, example types of data stored by computing system 104 may include authentication identifiers, ear-wearable device settings, settings for mobile applications (e.g., mobile applications running on user interface device 108, third-party computing device 106, or other devices), ear-wearable device serial numbers, receiver matrixes, fitting formulas, ear-wearable device firmware versions, audiogram data, demographic data, accessory data, location data, data log files, sensor data (e.g. heart rate data, step count data, exercise metrics, temperature data, user fall history, etc.).

As mentioned elsewhere in this disclosure, sharing data as part of a similar demographic group or competitive venture can increase success in wellness goals or faster acclimatization to wellness devices like ear-wearable device(s) 102 (e.g., hearing aids), fitness sensors, or heart-rate monitors. In some examples, a subset of the above-mentioned health-related data types may be shared among users of ear-wearable devices for the purpose of competition. For the purpose of competition, computing system 104 may anonymize the data unless explicit connections are made. Explicit connections may be made in the manner used by social networking services, such as Facebook, Instagram, and Twitter. Additionally, computing system 104 may enable users to view comparisons of their health-related data relative to health-related data of other users who have opted in. Furthermore, in some examples, computing system 104 may sort health-related data by age, sex, severity of hearing impairment, geographical location, self-identified level of activity, self-identified level of health, self-identified level of competitiveness, or other factors. In some examples, computing system 104 may sort the health-related data by performance. For instance, computing system 104 may enable user 112 to determine how user 112 compares to the top 10% of users of ear-wearable devices, how user 112 compares to the top 25% of users of ear-wearable devices, and so on. Furthermore, computing system 104 may enable competitions that last different amounts of time (e.g., daily challenges, weekly challenges, etc.).

Furthermore, computing system 104 may implement a reward structure for each competition. The reward structure may reward consistency in performance, such as rewarding “streaks” of positive data or use. In some examples, the reward structure may provide incentives that have value, like unlocking features, extending subscriptions, or lower insurance premiums. Furthermore, in some examples, computing system 104 may implement a system of rewarding consistency in which users are given end user(s) tokens or badges in the user interface for their performance. A form of competition, then, would be the ability to compare such rewards across users. For examples, users may be motivated to compare whether their competitor has more badges in total, or perhaps more coveted badges—such as for the longest streak of days with blood pressure in a healthy range, or the longest streak of days with the Brain or Body Score above some specified performance threshold. Another form of reward may include the awarding of points that could be used to access or “unlock” additional functionality or features of the ear-worn device(s). This granted access could be accomplished via a Field Firmware Update (FFU).

In some examples, computing system 104 may perform comparisons to like groups as part of enabling user 112 or third-party user 114 to compare health-related data of user 112. For instance, computing system 104 may filter health-related data of users according to demographic info (sex, age, hearing impairment, etc.) and use the filtered health-related data to generate comparison data. Furthermore, for purposes of competition, computing system 104 may perform comparisons of the health-related data of user 112 with selected, known persons, with representative groups of users, with anonymous competitors, or other types of people.

In some examples, health monitoring system 100 may implement a 2-factor authentication system to verify the identities of and devices associated with third-party users. For instance, in some examples, as part of indicating that a particular third-party user is authorized to access one or more health-related data types, user 112 may identify the particular third-party user by specifying a known mobile phone number or email address of the particular third-party user. Computing system 104 (or a computer associated with user 112) may then send a message containing a code to the mobile phone number (e.g., in the form of an SMS message) or an email to the email address. Prior to a third-party user being able to access the health-related data shared by user 112, computing system 104 may require the third-party user to provide the code.

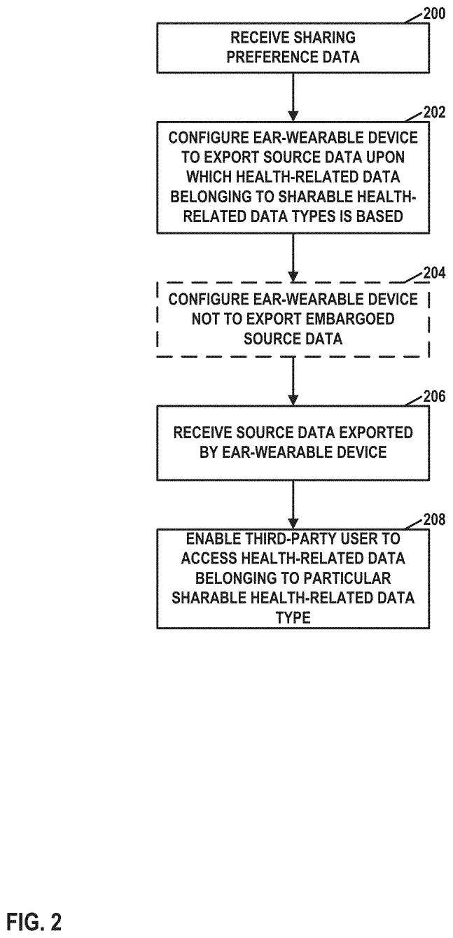

FIG. 2 is a flowchart illustrating an example operation of computing system 104 in accordance with a technique of this disclosure. In other examples, computing system 104 may perform more, fewer, or different actions than those shown in FIG. 2.

In the example of FIG. 2, computing system 104 receives sharing preference data (200). In some examples, computing system 104 may receive the sharing preference data via a user interface that comprises separate features for each respective health-related data type of a plurality of health-related data types. In such examples, for each respective health-related data type of the plurality of health-related data types, the features for the respective health-related data type enable specifying (e.g., by an interface user such as user 112, user 116, or another user) sharing preferences for the respective health-related data type of user 112 of ear-wearable device(s) 102. In some examples, computing system 104 may receive the sharing preference data, via computer-to-computer communication interface, from another computing system, such as a computing system of an insurance company or healthcare provider.

The sharing preference data indicate, for each respective health-related data type of the plurality of health-related data types: (i) whether user 112 of ear-wearable device(s) 102 allows sharing of health-related data belonging to the respective health-related data type, and (ii) if the user of the ear-wearable device allows sharing of health-related data belonging to the respective health-related data type, a set of one or more persons allowed by user 112 of ear-wearable device(s) 102 to access the health-related data belonging to the respective health-related data type.

As described elsewhere in this disclosure, a set of sharable health-related data types includes each respective health-related data type in the plurality of health-related data types for which the sharing preference data indicate that user 112 of ear-wearable device(s) 102 allows sharing of the health-related data belonging to the health-related data types. Similarly, a set of non-sharable health-related data types includes each respective health-related data type in the plurality of health-related data types for which the sharing preference data indicate that user 112 of ear-wearable device(s) 102 does not allow sharing of the health-related data belonging to the health-related data types.

Furthermore, in the example of FIG. 2, for each respective sharable health-related data type of the set of sharable health-related data types, computing system 104 may configure ear-wearable device(s) 102 to export source data upon which the health-related data belonging to the respective sharable health-related data type is based (202). For instance, computing system 104 may send to, and ear-wearable device(s) 102 may store, data identifying types of source data to export. Ear-wearable device(s) 102 may use this data when determining which types of source data to export. In some examples, computing system 104 does not configure ear-wearable device(s) 102 to export source data, but rather computing system 104 receives source data exported by ear-wearable device(s) 102 regardless of whether the sharable or non-sharable health-related data is based on the source data.

In some examples, computing system 104 may also configure ear-wearable device(s) 102 not to export embargoed source data (204). For instance, computing system 104 may send, and ear-wearable device(s) 102 may store, data indicating types of embargoed source data. In some examples, computing system 104 may configure ear-wearable device(s) 102 not to export embargoed source data by not sending data that indicate that ear-wearable device(s) 102 are to export the embargoed source data. For each respective sharable health-related data type of the set of sharable health-related data types, the health-related data belonging to the respective sharable health-related data type is not based on the embargoed source data, and health-related data belonging to one or more of the non-sharable health-related data types is based on the embargoed source data. In other examples, computing system 104 may export source data upon which non-shareable health-related data is based in order to enable user 112 to access the non-shareable health-related data without enabling any other user else to access non-shareable health-related data.

Computing system 104 may receive source data exported by the ear-wearable device(s) 102 (206). The source data exported by ear-wearable device(s) 102 comprises source data upon which health-related data belonging to the sharable health-related data types is based. In some examples, computing system 104 comprises a cloud-based infrastructure that includes computing devices remote from ear-wearable device(s) 102. For instance, computing system 104 may comprise one or more server devices in a data center. Ear-wearable device(s) 102 may communicate source data (e.g., device usage and health/wellness information) directly to a cloud-based infrastructure of computing system 104. For instance, ear-wearable device(s) 102 may communicate with computing system 104 via a wired- or wireless-network access point that is connected to the Internet.

In other examples, ear-wearable device(s) 102 communicate with computing system 104 indirectly through a mobile accessory wireless cloud infrastructure gateway device, such as a smartphone, tablet, bracelet, watch, or any type of body worn or handheld wireless gateway devices. In some examples, the gateway device comprises sensors or communicates with sensor devices 110 in order to generate particular types of source data even when user 112 is not wearing ear-wearable device(s) 102, such as during the night or when user 112 is in the bath or shower. One or more of the gateway devices may have a companion application installed thereon that may facilitate communication of source data (e.g., device usage data, health/wellness information, etc.) to computing system 104, which may store the source data in a cloud-accessible database. In some examples, user 116 or user 112 may selectively provide third-party users (e.g., professionals, physicians, relatives, etc.), such as third-party user 114, with access to health-related data of user 112 by configuring permission settings on the companion application, which may be accessible from their wireless cloud infrastructure gateway device having a display such as a smartphone, smart watch, or tablet.

Computing system 104 may enable a third-party user (e.g., third-party user 114) to access health-related data belonging to a particular sharable health-related data type (210). The health-related data belonging to the particular sharable health-related data type is based on the source data exported by ear-wearable device(s) 102. Third-party user 114 may be a user allowed by user 112 of ear-wearable device(s) 102 to access the health-related data belonging to the particular sharable health-related data type. In some examples, third-party computing device 106 may receive and store the health-related data.

In this way, health monitoring system 100 may allow third-party user 114 (e.g., a professional, physicians, friend, or relative) to remotely access device usage and health/wellness information or other health-related data based on the consent of user 112. The health-related data may include data regarding physical activity, cognitive fitness based on social activity and other factors, compliance with suggested health regimens or guidelines, and other physiological measurements collected by sensors built into ear-wearable device(s) 102, sensor devices 110, accessory devices, or other types of devices.

In some examples, computing system 104 may provide health-related data to third-party computing device 106 on a real time basis, on a periodic basis, in response to various types of events, or according to another type of updating regime. Similarly, third-party computing device 106 may present health-related data on a real time basis, on a periodic basis, in response to various types of events or according to another type of updating regime. In examples, where the updating regime is a periodic basis, computing system 104 may provide health-related data defined monitoring intervals of data, discrete event and score based running total data measurements.

In some examples, third-party computing device 106 may generate notifications or alerts based on health-related data. For instance, third-party computing device 106 may generate alerts when new health-related data regarding user 112 is available to user 114, when a level of a wellness measure of user 112 is outside of limits defined by user 112 or a third-party user 114, when a level of a wellness measure of user 112 has not been evaluated for a defined amount of time, or other under other circumstances. In any of the examples of this disclosure, an alert may be an app-based notification, an SMS message, a text message, an email message, or another type of indicator directed to third-party user 114.

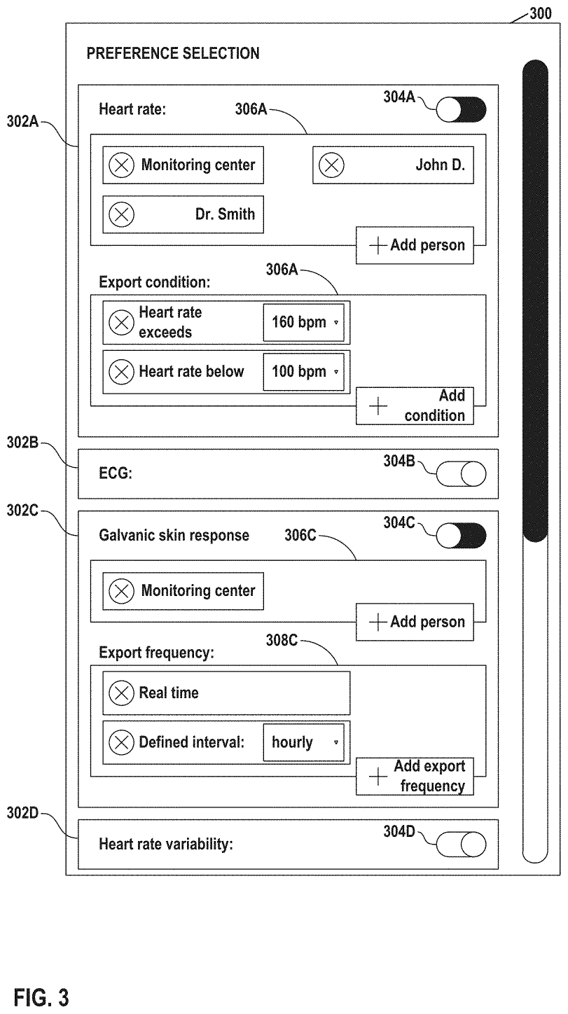

FIG. 3 is a conceptual diagram illustrating an example graphical user interface (GUI) 300 that comprises features for health-related data types, in accordance with one or more techniques of this disclosure. In the example of FIG. 3, GUI 300 includes a set of features 302A-302D (collectively, “features 302”) that correspond to different health-related data types. Particularly, in the example of FIG. 3, feature 302A corresponds to heart rate, feature 302B corresponds to ECG, feature 302C corresponds to galvanic skin response, and feature 302D corresponds to heart rate variability. Each of features 302 includes one of respective toggle controls 304A-304D (collectively, “toggle controls 304”) that control whether user 112 has permitted sharing of the corresponding health-related data type.

Furthermore, for each of toggle controls 304, if the toggle control is switched to indicate user 112 has permitted sharing of the corresponding health-related data type, the feature 302 corresponding to the toggle control may include a feature (e.g., user-selection feature 306A, user-selection feature 306C) that enables user selection of third-party users that are permitted to access the corresponding health-related data type.

Additionally, for each of toggle controls 304, if the toggle control is switched to indicate user 112 has permitted sharing of the corresponding health-related data type, the feature 302 corresponding to the toggle control may include one or more features that are specific to the health-related data type. For instance, in the example of FIG. 3, feature 302A includes a feature 308A that enables a user to select conditions under which ear-wearable device(s) 102 export source data upon which the heart rate data is based. Thus, in any of the examples of this disclosure, computing system 104 may receive (e.g., via a user interface, such as GUI 300), export condition preference data that indicate an export condition preference for a sharable health-related data type. In such examples, computing system 104 may configure ear-wearable device(s) 102 such that ear-wearable device(s) 102 exports source data upon which the particular sharable health-related data type is based when a condition specified by the export condition preference for the particular sharable health-related data type is satisfied.

Additionally, in the example of FIG. 3, feature 302C includes a feature 308C that enables a user to select a frequency at which ear-wearable device(s) 102 export source data upon which the galvanic skin response data is based. For instance, in the example of FIG. 3, feature 308C is currently configured for real time export of the source data and also export of the source data on an hourly basis.

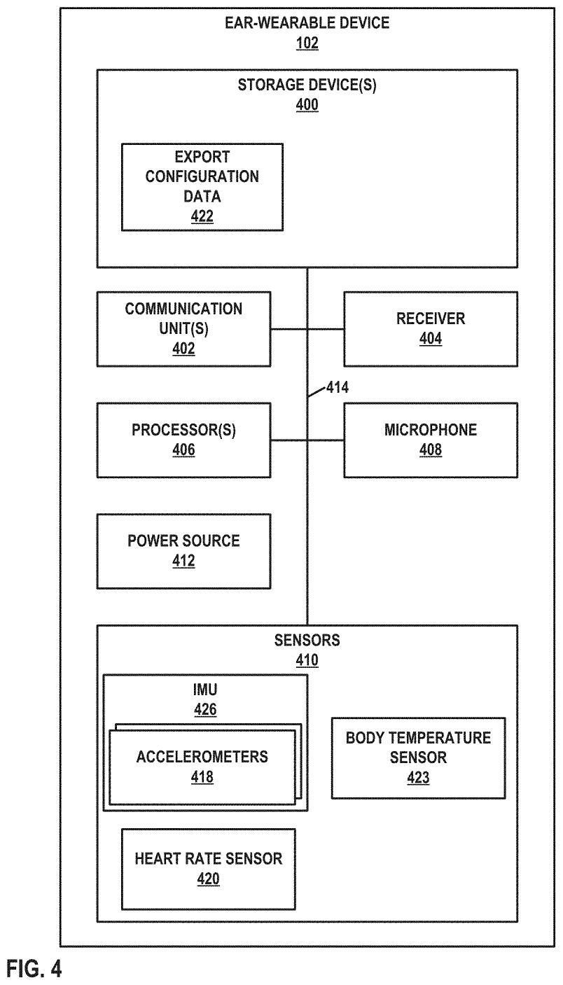

FIG. 4 is a block diagram illustrating example components of ear-wearable device 102, in accordance with one or more techniques of this disclosure. In the example of FIG. 4, ear-wearable device 102 comprises one or more storage device(s) 400, a communication unit(s) 402, a receiver 404, one or more processor(s) 406, a microphone 408, a set of sensors 410, a power source 412, and one or more communication channels 414. Communication channels 414 provide communication between storage device(s) 400, communication unit(s) 402, receiver 404, processor(s) 406, a microphone 408, and sensors 410. Components 400, 402, 404, 406, 408, and 410 may draw electrical power from power source 412. Power source 412 may be a battery, capacitor, or other type of component for storing energy.

In the example of FIG. 4, sensors 410 include one or more accelerometers 418. Furthermore, in examples such as that of FIG. 4, an inertial measurement unit (IMU) 426 includes one or more of accelerometers 418. The IMU 426 may use signals generated by accelerometers 418 for various purposes. For example, the IMU 426 may use the signals generated by accelerometers 418 to count the number of steps that user 112 of ear-wearable device 102 has taken.

Additionally, in the example of FIG. 4, sensors 410 also include a heart rate sensor 420 and a body temperature sensor 423. In other examples, ear-wearable device 102 may include more, fewer, or different components. For instance, in other examples, ear-wearable device 102 does not include particular sensors shown in the example of FIG. 4. In some examples, heart rate sensor 420 comprises a visible light sensor and/or a pulse oximetry sensor.

Storage device(s) 400 may store data. Storage device(s) 400 may comprise volatile memory and may therefore not retain stored contents if powered off. Examples of volatile memories may include random access memories (RAM), dynamic random access memories (DRAM), static random access memories (SRAM), and other forms of volatile memories known in the art. Storage device(s) 400 may further be configured for long-term storage of information as non-volatile memory space and retain information after power on/off cycles. Examples of non-volatile memory configurations may include magnetic hard discs, optical discs, floppy discs, flash memories, or forms of electrically programmable memories (EPROM) or electrically erasable and programmable (EEPROM) memories.

Communication unit(s) 402 may enable ear-wearable device 102 to send data to and receive data from one or more other computing devices. For example, communication unit(s) 402 may enable ear-wearable device 102 to send data to and receive data from a computing device of computing system 104, a gateway device, or another type of device. Communication unit(s) 402 may use one or more of various types of wireless technology to communicate. For instance, communication unit(s) 402 may use Bluetooth, 3G, 4G, 4G LTE, ZigBee, WiFi, Near-Field Magnetic Induction (NFMI), or another communication technology. In some examples, communication unit(s) 402 also enable ear-wearable device 102 to communicate with another ear-wearable device worn by user 112.

Receiver 404 comprises one or more speakers for generating audible sound. Microphone 408 detects incoming sound and generates an electrical signal (e.g., an analog or digital electrical signal) representing the incoming sound. Processor(s) 406 may process the signal generated by microphone 408 to enhance, amplify, or cancel-out particular channels within the incoming sound. Processor(s) 406 may then cause receiver 404 to generate sound based on the processed signal. In some examples, processor(s) 406 include one or more digital signal processors (DSPs).

Processor(s) 406 may cause communication unit(s) 402 to transmit one or more of various types of data. For example, processor(s) 406 may cause communication unit(s) 402 to transmit data to computing system 104. Furthermore, communication unit(s) 402 may receive audio data from computing system 104 and processor(s) 406 may cause receiver 404 to output sound based on the audio data.

In some examples, ear-wearable device 102 is a “plug-n-play” type of device. In some examples, ear-wearable device 102 is programmable to help the user manage things like wind noise. Furthermore, in some examples, ear-wearable device 102 comprises a custom earmold or a standard receiver module at the end of a RIC cable. The additional volume in a custom earmold may allow room for components such as sensors (accelerometers, heartrate monitors, temp sensors), a woofer-tweeter, (providing richer sound for music aficionados), and an acoustic valve that provides occlusion when desired. In some examples, a six conductor RIC cable is used for in ear-wearable devices with sensors, woofer-tweeters, and/or acoustic valves.

In the example of FIG. 4, storage device(s) 400 may include export configuration data 422. Export configuration data 422 indicates which types of source data to export to computing system 104. Thus, export configuration data 422 may indicate that ear-wearable device 102 is to export source data upon which sharable health-related data types is based and not to export embargoed source data. Processor(s) 406 may use communication unit(s) 402 to export source data to computing system 104. Furthermore, in some examples, processor(s) 406 may generate source data based on data generated by sensors (e.g., sensors 410, sensors of sensor devices 110), such as by applying one or more algorithms to the data generated by the sensors. In some examples, the source data is untransformed data generated by the sensors.

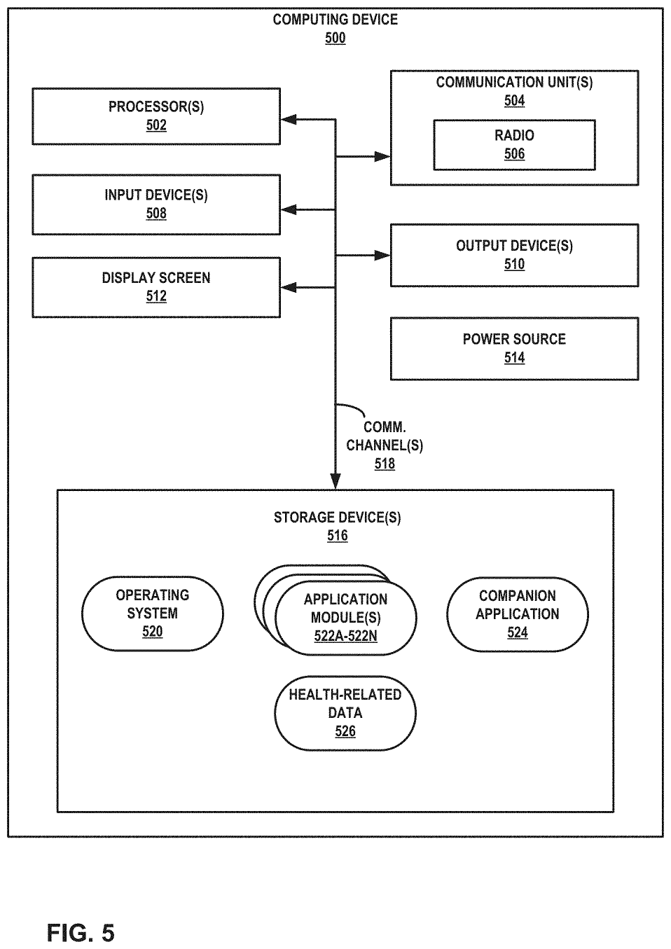

FIG. 5 is a block diagram illustrating example components of a computing device 500 of computing system 104, in accordance with one or more techniques of this disclosure. Computing device 500 may be user interface device 108, third-party computing device 106, or another computing device. FIG. 5 illustrates only one particular example of computing device 500, and many other example configurations of computing device 500 exist.

As shown in the example of FIG. 5, computing device 500 includes one or more processor(s) 502, one or more communication unit(s) 504, one or more input device(s) 508, one or more output device(s) 510, a display screen 512, a power source 514, one or more storage device(s) 516, and one or more communication channels 518. Computing device 500 may include other components. For example, computing device 500 may include physical buttons, microphones, speakers, communication ports, and so on. Communication channel(s) 518 may interconnect each of components 502, 504, 508, 510, 512, and 516 for inter-component communications (physically, communicatively, and/or operatively). In some examples, communication channel(s) 518 may include a system bus, a network connection, an inter-process communication data structure, or any other method for communicating data. Power source 514 may provide electrical energy to components 502, 504, 508, 510, 512 and 516.

Storage device(s) 516 may store information required for use during operation of computing device 500. In some examples, storage device(s) 516 have the primary purpose of being a short term and not a long-term computer-readable storage medium. Storage device(s) 516 may be volatile memory and may therefore not retain stored contents if powered off. Storage device(s) 516 may further be configured for long-term storage of information as non-volatile memory space and retain information after power on/off cycles. In some examples, processor(s) 502 on computing device 500 read and may execute instructions stored by storage device(s) 516.

Computing device 500 may include one or more input device(s) 508 that computing device 500 uses to receive user input. Examples of user input include tactile, audio, and video user input. Input device(s) 508 may include presence-sensitive screens, touch-sensitive screens, mice, keyboards, voice responsive systems, microphones or other types of devices for detecting input from a human or machine.

Communication unit(s) 504 may enable computing device 500 to send data to and receive data from one or more other computing devices (e.g., via a communications network, such as a local area network or the Internet). For instance, communication unit(s) 504 may be configured to receive source data exported by ear-wearable device(s) 102, receive comment data generated by user 112 of ear-wearable device(s) 102, receive and send request data, receive and send messages, and so on. In some examples, communication unit(s) 504 may include wireless transmitters and receivers that enable computing device 500 to communicate wirelessly with the other computing devices. For instance, in the example of FIG. 5, communication unit(s) 504 include a radio 506 that enables computing device 500 to communicate wirelessly with other computing devices, such as ear-wearable device 102 (FIG. 1, FIG. 4). Examples of communication unit(s) 504 may include network interface cards, Ethernet cards, optical transceivers, radio frequency transceivers, or other types of devices that are able to send and receive information. Other examples of such communication units may include Bluetooth, 3G, and WiFi radios, Universal Serial Bus (USB) interfaces, etc. Computing device 500 may use communication unit(s) 504 to communicate with one or more ear-wearable devices (e.g., ear-wearable device 102 (FIG. 1, FIG. 4)). Additionally, computing device 500 may use communication unit(s) 504 to communicate with one or more other remote devices.

Output device(s) 510 may generate output. Examples of output include tactile, audio, and video output. Output device(s) 510 may include presence-sensitive screens, sound cards, video graphics adapter cards, speakers, liquid crystal displays (LCD), or other types of devices for generating output.

Processor(s) 502 may read instructions from storage device(s) 516 and may execute instructions stored by storage device(s) 516. Execution of the instructions by processor(s) 502 may configure or cause computing device 500 to provide at least some of the functionality ascribed in this disclosure to computing device 500. As shown in the example of FIG. 5, storage device(s) 516 include computer-readable instructions associated with operating system 520, application modules 522A-522N (collectively, “application modules 522”), and a companion application 524. Additionally, in the example of FIG. 5, storage device(s) 516 may store health-related data 526.

Execution of instructions associated with operating system 520 may cause computing device 500 to perform various functions to manage hardware resources of computing device 500 and to provide various common services for other computer programs. Execution of instructions associated with application modules 522 may cause computing device 500 to provide one or more of various applications (e.g., “apps,” operating system applications, etc.). Application modules 522 may provide particular applications, such as text messaging (e.g., SMS) applications, instant messaging applications, email applications, social media applications, text composition applications, and so on.

Execution of instructions associated with companion application 524 may cause computing device 500 to perform one or more of various functions described in this disclosure with respect to computing system 104 (FIG. 1). For example, execution of instructions associated with companion application 524 may cause computing device 500 to configure communication unit(s) 504 to receive data from ear-wearable device(s) 102. Although not explicitly recited here for the sake of brevity, instructions of companion application 524 may cause computing device 500 to perform one or more of various actions of computing system 104 described elsewhere in this disclosure. In some examples, companion application 524 is an instance of a web application or server application. In some examples, such as examples where computing device 500 is a mobile device or other type of computing device, companion application 524 may be a native application.

Companion application 524 may store one or more of various types of health-related data 526. In some examples, companion application 524 may store health-related data 526 in storage device(s) 516 a database for storing health-related data. Companion application 524 may retrieve health-related data 526 from storage device(s) 516 to enable authorized third-party users to access the health-related data.

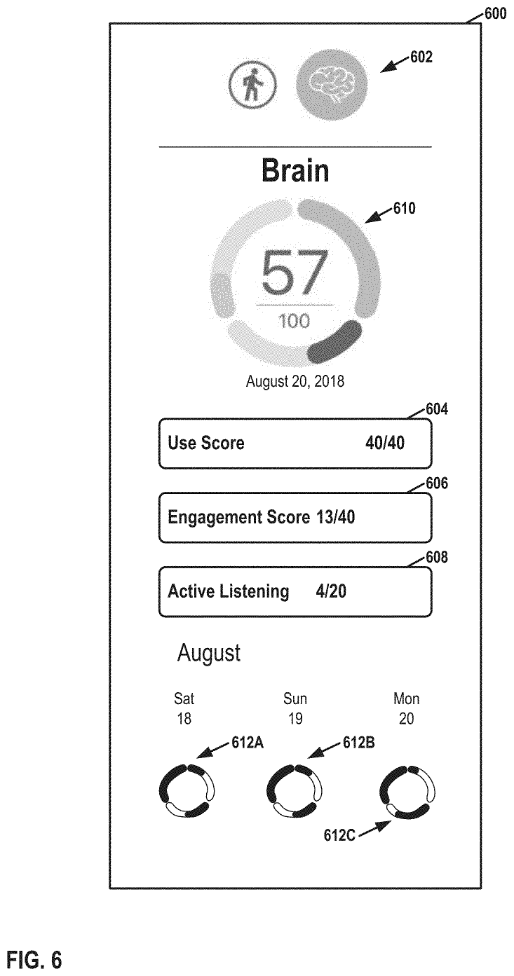

FIG. 6 is an example GUI 600 for display of a cognitive benefit measure in accordance with one or more techniques of this disclosure. In the example of FIG. 6, GUI 600 includes controls 602 that allow a user to switch between a user interface for display of the cognitive benefit measure and a user interface for display of a body fitness measure. GUI 600 is an example of a type of GUI that third-party computing device 106 or a computing device associated with user 112 may output for display in order to enable third-party user 114 or user 112 to access health-related data. In this example of FIG. 6, the health-related data is a brain score (i.e., a cognitive benefit measure) of user 112.

In the example of FIG. 6, the cognitive benefit measure is based on a use score sub-component, an engagement score sub-component, and an active listening sub-component. Feature 604 of GUI 600 indicates a value of the use score sub-component. Feature 606 of GUI 600 indicates a value of the engagement score sub-component. Feature 608 of GUI 600 indicates a value of the active listening sub-component. In features 604, 606, and 608 of GUI 600, the value before the “/” mark indicates a current value of the sub-component and the value after the “/” mark indicates a goal for the sub-component.

Furthermore, in the example of FIG. 6, GUI 600 includes a circular diagram 610 having segments corresponding to the sub-components of the cognitive benefit measure. Each of the segments is filled in an amount proportional to the wearer's progress toward meeting the goals for the sub-components of the cognitive benefit measure. Additionally, circular diagram 610 may include a numerical value indicating the wearer's cognitive benefit measure (e.g., 57 in the example of FIG. 6) and a numerical value indicating the wearer's cognitive benefit measure goal for the cognitive benefit measure (e.g., 100 in the example of FIG. 6). The wearer's cognitive benefit measure goal is the wearer's goal for the cognitive benefit measure.

GUI 600 also includes historical icons 612A, 612B, and 612C (collectively, “historical icons 612”). In the example of FIG. 6, like circular diagram 610, historical icons 612 include segments with filled portions corresponding to indicate the wearer's progress toward meeting the goals for the sub-components on previous days, e.g., Saturday, Sunday and Monday in the example of FIG. 6. In response to receiving an indication of user selection of one of historical icons 612, computing system 104 may output for display a GUI having more details regarding the wearer's cognitive benefit measure for the day corresponding to the selected historical icon.

In this disclosure, ordinal terms such as “first,” “second,” “third,” and so on, are not necessarily indicators of positions within an order, but rather may be used to distinguish different instances of the same thing. Examples provided in this disclosure may be used together, separately, or in various combinations.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processing circuits to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, cache memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Functionality described in this disclosure may be performed by fixed function and/or programmable processing circuitry. For instance, instructions may be executed by fixed function and/or programmable processing circuitry. Such processing circuitry may include one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structure or any other structure suitable for implementation of the techniques described herein. In addition, in any of the examples of this disclosure, the functionality described herein may be provided within dedicated hardware and/or software modules. Also, the techniques could be fully implemented in one or more processing circuits or logic elements. Processing circuits may be coupled to other components in various ways. For example, a processing circuit may be coupled to other components via an internal device interconnect, a wired or wireless network connection, or another communication medium.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

Various examples have been described. These and other examples are within the scope of the following claims.

What is claimed is:

1. A method comprising:
receiving, by a computing system, sharing preference data, wherein:
the sharing preference data indicate, for each respective health-related data type of a plurality of health-related data types:
(i) whether a user of an ear-wearable device allows sharing of health-related data belonging to the respective health-related data type, and
(ii) if the user of the ear-wearable device allows sharing of health-related data belonging to the respective health-related data type, a set of one or more users that are allowed by the user of the ear-wearable device to access the health-related data belonging to the respective health-related data type,
a set of sharable health-related data types includes each respective health-related data type in the plurality of health-related data types for which the sharing preference data indicate that the user of the ear-wearable device allows sharing of the health-related data belonging to the health-related data types,
a set of non-sharable health-related data types includes each respective health-related data type in the plurality of health-related data types for which the sharing preference data indicate that the user of the ear-wearable device does not allow sharing of the health-related data belonging to the health-related data types;
for each respective sharable health-related data type of the set of sharable health-related data types, configuring, by the computing system, the ear-wearable device to export source data upon which the health-related data belonging to the respective sharable health-related data type is based;
receiving, by the computing system, source data exported by the ear-wearable device; and
enabling, by the computing system, a third-party user to access health-related data belonging to a particular sharable health-related data type, wherein:
the health-related data belonging to the particular sharable health-related data type is based on the source data exported by the ear-wearable device, and
the third-party user is a user allowed by the user of the ear-wearable device to access the health-related data belonging to the particular sharable health-related data type.

2. The method of claim 1, further comprising:
receiving, by the computing system, comment data generated by the user of the ear-wearable device, wherein the comment data is associated with the particular sharable health-related data type; and
for each respective sharable health-related data type of the set of sharable health-related data types:
determining, by the computing system, whether there is comment data associated with the respective sharable health-related data type; and
in response to determining that there is comment data associated with the respective sharable health-related data type, generating, by the computing system, health-related data belonging to the respective sharable health related data type based on the source data exported by the ear-wearable device, wherein the health-related data belonging to the respective sharable health-related data type includes the comment data associated with the respective sharable health-related data type.

3. The method of claim 1, further comprising:
receiving, by the computing system, first request data from a computing device associated with the third-party user, the first request data comprising a request for updated health-related data belonging to the particular sharable health-related data type;
in response to receiving the first request data, sending, by the computing system, second request data to the ear-wearable device;
receiving, by the computing system, updated source data from the ear-wearable device in response to the second request data;
generating, by the computing system, the updated health-related data based on the updated source data; and
enabling, by the computing system, the third-party user to access the updated health-related data.

4. The method of claim 1, wherein the method further comprises:
receiving, by the computing system, export condition preference data that indicate an export condition preference for the particular sharable health-related data type; and
configuring, by the computing system, the ear-wearable device such that the ear-wearable device exports source data upon which the particular sharable health-related data type is based when a condition specified by the export condition preference for the particular sharable health-related data type is satisfied.

5. The method of claim 1, further comprising:
receiving, by the computing system, message data comprising a message from the third-party user to the user of the ear-wearable device; and
sending, by the computing system, the message data to a computing device associated with the user of the ear-wearable device.

6. The method of claim 1, further comprising configuring, by the computing system, the ear-wearable device not to export embargoed source data, wherein for each respective sharable health-related data type of the set of sharable health-related data types, the health-related data belonging to the respective sharable health-related data type is not based on the embargoed source data, and health-related data belonging to one or more of the non-sharable health-related data types is based on the embargoed source data.

7. The method of claim 1, wherein:
receiving the sharing preference data comprises, receiving, by the computing system, the sharing preference data via a user interface, wherein the user interface comprises separate features for each respective health-related data type of the plurality of health-related data types, and
for each respective health-related data type of the plurality of health-related data types, the features for the respective health-related data type enable specifying sharing preferences for the respective health-related data type of the user of the ear-wearable device.

8. A computing system comprising:
one or more communication units; and
one or more processing circuits configured to:
receive sharing preference data, wherein:
the sharing preference data indicate, for each respective health-related data type of a plurality of health-related data types:
(i) whether a user of an ear-wearable device allows sharing of health-related data belonging to the respective health-related data type, and
(ii) if the user of the ear-wearable device allows sharing of health-related data belonging to the respective health-related data type, a set of one or more users that are allowed by the user of the ear-wearable device to access the health-related data belonging to the respective health-related data type,
a set of sharable health-related data types includes each respective health-related data type in the plurality of health-related data types for which the sharing preference data indicate that the user of the ear-wearable device allows sharing of the health-related data belonging to the health-related data types,
a set of non-sharable health-related data types includes each respective health-related data type in the plurality of health-related data types for which the sharing preference data indicate that the user of the ear-wearable device does not allow sharing of the health-related data belonging to the health-related data types;
for each respective sharable health-related data type of the set of sharable health-related data types, configure the ear-wearable device to export source data upon which the health-related data belonging to the respective sharable health-related data type is based,
wherein the one or more communication units are configured to receive source data exported by the ear-wearable device; and
wherein the one or more processing circuits are further configured to enable a third-party user to access health-related data belonging to a particular sharable health-related data type, wherein:
the health-related data belonging to the particular sharable health-related data type is based on the source data exported by the ear-wearable device, and
the third-party user is a user allowed by the user of the ear-wearable device to access the health-related data belonging to the particular sharable health-related data type.

9. The computing system of claim 8, wherein:
the one or more communication units are further configured to receive comment data generated by the user of the ear-wearable device, wherein the comment data is associated with the particular sharable health-related data type, and
the one or more processing circuits are further configured to, for each respective sharable health-related data type of the set of sharable health-related data types:
determine whether there is comment data associated with the respective sharable health-related data type; and
in response to determining that there is comment data associated with the respective sharable health-related data type, generate health-related data belonging to the respective sharable health related data type based on the source data exported by the ear-wearable device, wherein the health-related data belonging to the respective sharable health-related data type includes the comment data associated with the respective sharable health-related data type.

10. The computing system of claim 8, wherein:
the one or more communication units are configured to:
receive first request data from a computing device associated with the third-party user, the first request data comprising a request for updated health-related data belonging to the particular sharable health-related data type;
in response to receiving the first request data, send second request data to the ear-wearable device; and
receive updated source data from the ear-wearable device in response to the second request data;
the one or more processing circuits are configured to:
generate the updated health-related data based on the updated source data; and
enable the third-party user to access the updated health-related data.

11. The computing system of claim 8, wherein the one or more processing circuits are further configured to:
receive export condition preference data that indicate an export condition preference for the particular sharable health-related data type; and
configure the ear-wearable device such that the ear-wearable device exports source data upon which the particular sharable health-related data type is based when a condition specified by the export condition preference for the particular sharable health-related data type is satisfied.

12. The computing system of claim 8, wherein the one or more communication units are further configured to:
receive message data comprising a message from the third-party user to the user of the ear-wearable device; and
send the message data to a computing device associated with the user of the ear-wearable device.

13. The computing system of claim 8, wherein the one or more processing circuits are further configured to:
configure the ear-wearable device not to export embargoed source data, wherein for each respective sharable health-related data type of the set of sharable health-related data types, the health-related data belonging to the respective sharable health-related data type is not based on the embargoed source data, and health-related data belonging to one or more of the non-sharable health-related data types is based on the embargoed source data.

14. The computing system of claim 8, wherein the one or more processing circuits are configured to:
receive the sharing preference data via a user interface, wherein the user interface comprises separate features for each respective health-related data type of the plurality of health-related data types, and
for each respective health-related data type of the plurality of health-related data types, the features for the respective health-related data type enable specifying sharing preferences for the respective health-related data type of the user of the ear-wearable device.

15. A computer-readable data storage medium having instructions stored thereon that, when executed, cause one or more processing circuits of a computing system to:
receive sharing preference data, wherein:
the sharing preference data indicate, for each respective health-related data type of a plurality of health-related data types:
(i) whether a user of an ear-wearable device allows sharing of health-related data belonging to the respective health-related data type, and
(ii) if the user of the ear-wearable device allows sharing of health-related data belonging to the respective health-related data type, a set of one or more users that are allowed by the user of the ear-wearable device to access the health-related data belonging to the respective health-related data type,
a set of sharable health-related data types includes each respective health-related data type in the plurality of health-related data types for which the sharing preference data indicate that the user of the ear-wearable device allows sharing of the health-related data belonging to the health-related data types,
a set of non-sharable health-related data types includes each respective health-related data type in the plurality of health-related data types for which the sharing preference data indicate that the user of the ear-wearable device does not allow sharing of the health-related data belonging to the health-related data types;
for each respective sharable health-related data type of the set of sharable health-related data types, configure the ear-wearable device to export source data upon which the health-related data belonging to the respective sharable health-related data type is based;
receive source data exported by the ear-wearable device; and
enable a third-party user to access health-related data belonging to a particular sharable health-related data type, wherein:
the health-related data belonging to the particular sharable health-related data type is based on the source data exported by the ear-wearable device, and
the third-party user is a user allowed by the user of the ear-wearable device to access the health-related data belonging to the particular sharable health-related data type.
